(19) United States
(12) Patent Application Publication
(10) Pub. No.: US 2008/0194944 A1
(43) Pub. Date: Aug. 14, 2008

(54) SUPPRESSION OF BACKGROUND TISSUES IN MRA IMAGES

(76) Inventor: Robert R. Edelman, Highland Park, IL (US)

Correspondence Address: QUARLES & BRADY LLP, 411 E. WISCONSIN AVENUE, SUITE 2040, MILWAUKEE, WI, 53202-4497, US

(21) Appl. No.: 11/834,754

(22) Filed: Aug. 7, 2007

Related U.S. Application Data
(60) Provisional application No. 60/915,781, filed on May 3, 2007.
(60) Provisional application No. 60/836,164, filed on Aug. 8, 2006.

(51) Int. Cl. A61B 5/055

(52) U.S. Cl. 600/420

(57) ABSTRACT

Background tissue signals are suppressed in an MRA image by using a contrast agent that shortens the T₁ relaxation and chemically shifts the tissue spins of interest. An imaging pulse sequence used to acquire the image data is tuned to the off-resonance tissue spins of interest and saturation pulse sequences are interleaved with the imaging pulse sequences to selectively suppress signals from on-resonance background tissues.

### CROSS-REFERENCE TO RELATED APPLICATIONS

This application is based on U.S. Provisional Patent Application Ser. No. 60/836,164 filed on Aug. 8, 200 entitled “SUPPRESSION OF BACKGROUND TISSUES IN MRA IMAGES” and “U.S. Provisional Patent Application Ser. No. 60/915,781 filed on May 3, 2007 entitled “MAGNETIC RESONANCE IMAGE ACQUISITION WITH SUPPRESSION OF BACKGROUND TISSUES AND RF WATER EXCITATION AT OFFSET FREQUENCY.”

### BACKGROUND OF THE INVENTION

The field of the invention is magnetic resonance imaging methods and systems and particularly magnetic resonance angiography.

The ability to depict anatomy and pathology by magnetic resonance (MRI) is critically dependent on the contrast, or difference in signal intensities between the target and background tissues. In order to maximize contrast, it is necessary to suppress the signal intensities of the background tissues. For instance, small blood vessels are much better depicted by the technique of MR angiography when the signal intensities of fat and muscle (background tissues) are minimized.

Magnetic resonance angiography (MRA) uses the nuclear magnetic resonance (NMR) phenomenon to produce images of the human vasculature. When a substance such as human tissue is subjected to a uniform magnetic field (polarizing field B₀), the individual magnetic moments of the spins in the tissue attempt to align with this polarizing field, but precess about it in random order at their characteristic Larmor frequency. If the substance, or tissue, is subjected to a magnetic field (excitation field B₁) which is in the x-y plane and which is near the Larmor frequency, the net aligned moment, M_{z}, may be rotated, or “tipped”, into the x-y plane to produce a net transverse magnetic moment Mₜ. A signal is emitted by the excited spins, and after the excitation signal B₁ is terminated, this signal may be received and processed to form an image.

When utilizing these signals to produce images, magnetic field gradients (Gₓ G_{y} and G_{z}) are employed. Typically, the region to be imaged is scanned by a sequence of measurement cycles in which these gradients vary according to the particular localization method being used. Each measurement is referred to in the art as a “view” and the number of views determines the resolution of the image. The resulting set of received NMR signals, or views, or k-space samples, are digitized and processed to reconstruct the image using one of many well known reconstruction techniques. The total scan time is determined in part by the number of measurement cycles, or views, that are acquired for an image, and therefore, scan time can be reduced at the expense of image resolution by reducing the number of acquired views.

To enhance the diagnostic capability of MRA a contrast agent such as gadolinium can be injected into the patient prior to the MRA scan. As described in U.S. Pat. No. 5,417,213 contrast enhanced (CE) MRA attempts to acquire the central k-space views at the moment the bolus of contrast agent is flowing through the vasculature of interest. Collection of the central lines of k-space during peak arterial enhancement is key to the success of a CEMRA exam. If the central lines of k-space are acquired prior to the arrival of contrast, severe image artifacts can limit the diagnostic information in the image. Alternatively, arterial images acquired after the passage of the peak arterial contrast are sometimes obscured by the enhancement of veins. In many anatomic regions, such as the carotid or renal arteries, the separation between arterial and venous enhancement can be as short as 6 seconds.

The ability to time the arrival of contrast in the vasculature of interest varies considerably and it is helpful in many applications to acquire a series of MRA images in a dynamic study which depicts the separate enhancement of arteries and veins. Such a temporal series of images is also useful for observing delayed vessel filling patterns caused by disease. This requirement has been partially addressed by acquiring a series of time resolved images using a 3D “Fourier” acquisition as described by Korosec F., Frayne R, Grist T., Mistretta C., “Time-Resolved Contrast-Enhanced 3D MR Angiography”, Magn. Reson. Med. 1996; 36:345-351 and in U.S. Pat. No. 5,713,358. More recently, time-resolved MRA images have been acquired using a three-dimensional projection reconstruction method as described in U.S. Pat. No. 6,487,435 entitled “Magnetic Resonance Angiography Using Undersampled 3D Projection Imaging”.

With CEMRA image studies the usual practice is to acquire at least one image prior to the injection of contrast into the patient. This pre-contrast image is used as a mask that is subtracted from the contrast enhanced images to remove the signal from surrounding non-vascular tissues and fat. While this technique can be very effective, it does have two disadvantages. First, it extends the time of the scan and it requires that the patient be immobilized so that the mask image is precisely registered with the contrast enhanced images from which it is subtracted. Any misregistration results in distracting image artifacts that may interfere with the diagnostic utility of the angiogram. The subtraction of two images also increases the standard deviation of the noise signal, reducing the signal-to-noise ratio (SNR) by the square root of 2.

A unique property of magnetic resonance imaging (MRI) is the ability to selectively image different chemical species by virtue of what is known as the chemical shift phenomenon. For example, in the human body fat exhibits a Larmor resonance which is separated from the water resonance by approximately 3.5 ppm. At a field strength of 2 Tesla this equates to a frequency separation of about 280 Hz in the NMR spectrum. Silicone also exhibits a chemical shift of approximately 5 ppm. This chemical shift has been exploited by a number of different techniques used to suppress signals from undesired tissues or to enhance signals from target tissues.

Suppression Methods

Techniques of fat suppression, generally referred to as FATSAT, are well known in the art. Fat suppression is usually achieved by placing a narrow band spectral rf suppression pulse before the imaging sequence. This pre-pulse is quickly followed by the imaging sequence so that the fat protons do not have time to relax back to their equilibrium magnetization which remains dispersed (saturated) and unable to contribute signal to the image.

FIG. 2 is a graph illustrating a prior art 3D gradient-echo motion compensated sequence having a frequency selective simple gaussian FATSAT spectral rf suppression pulse which is centered on the fat frequency. It is comprised of a presaturation sequence indicated at T_{P} and an imaging sequence indicated at TR. The graph labeled RF includes a gaussian spectral rf suppression pre-pulse 2 produced during the presaturation sequence followed by an imaging rf pulse 3 having a flip angle a produced during the imaging sequence.

The graphs labeled Gs, Ge and Gr represent the slice selection gradient, the phase encoding gradient and the readout gradient sequences, respectively. The slice selection gradient sequence includes a three-lobed motion compensated gradient 5, a phase encoding gradient 6 and a rewinder gradient 7 produced during the imaging sequence. A gradient pulse 4 is a spoiler pulse which is produced during the presaturation sequence Tp. The phase encoding gradient sequence Ge includes a phase encoding gradient 8 and a rewinder gradient 9. The readout gradient sequence includes a readout gradient 13, and during this readout pulse an NMR signal 11 is acquired.

FIG. 3 is a graph 15 representing a prior art NMR spectrum of fat and water protons on which a fat gaussian spectral suppression pulse is superimposed as indicated by graph 17. The horizontal axis represents the chemical shift in parts per million (PPM) units and the line labeled W indicates the peak absorption of the water protons at 0 ppm. The line labeled F indicates the peak of absorption of the fat protons which is shifted by 3.5 ppm relative to the peak absorption of the water protons. The gaussian suppression pulse 2 in the pulse sequence of FIG. 2 is centered at the peak F and will thus selectively excite the fat protons without substantial excitation of the water protons. Suppression pulses using the Sinc function are also known in the art.

Gaussian and Sinc type suppression pulses are required to be long in duration in order to achieve a suitably narrow spectral selection. At a field strength of 2 Tesla, for example, a typical Sinc suppression pulse may take up to 26 ms.

FIG. 4 is a graph illustrating a prior art 3D gradient-echo imaging sequence having a frequency selective binomial FATSAT spectral suppression presaturation pulse 21 which is centered on the water frequency. The use of binomial pulse suppression techniques is disclosed in an article appearing in The Journal of Magnetic Resonance, entitled “Solvent Suppression in Fourier Transform Nuclear Magnetic Resonance” by P. J. Hore (Vol. 55, 1983, pp. 283-300) incorporated herein by reference. The 1-3-3-1 binomial suppression pulse 21 includes four sub-pulses 21A, 21B, 21C and 21D which are separated from each other by a pulse separation interval τ. By choosing the appropriate pulse separation interval τ (dependent upon field strength and chemical species) the binomial pulse 21 exhibits a null excitation at the water frequency and rises to a 90° excitation at the fat frequency. This is illustrated by graph 41 in FIG. 5 which depicts the theoretical excitation spectrum of a prior art 1-3-3-1 binomial suppression pulse as a function of frequency offset from the transmitter frequency. The vertical axis of the graph represents the transverse magnetization M_{xy} wherein full scale corresponds to complete conversion of the longitudinal magnetization into transverse plane magnetization. The transverse magnetization curve 41 has a flat excitation null 39 around the transmitter frequency which is tuned to water.

The binomial pulse sequence 21 has a total duration of approximately 5.4 ms at 2 Tesla. This is shorter than the gaussian pulse 2 of FIG. 2, but it requires a high RF power because of the short “hard” pulses that are required.

Suppression techniques in a presaturation sequence generally extend the minimum TR that can be used and result in a reduction in the number of slices that can be imaged in a multi-slice acquisition. Presaturation methods are also limited when short TR's are required since rapid, repeated, and incomplete, saturation of the fat frequency inevitably leads to a build up of coherent fat signal resulting in image artifacts.

Spectral-Spatial Excitation Methods

Methods of Spectral-Spatial Excitation use a carefully designed RF modulation in the presence of an oscillating gradient to excite the target tissues. The result is a simultaneous selection along one spatial axis and the desired frequency spectrum. The use of Spectral-spatial excitation is disclosed in an article appearing in Magnetic Resonance in Medicine, entitled “Simultaneous Spatial and Spectral selective Excitation” by Craig H. Meyer et al. (Vol. 15, 1990, pp. 287-304), incorporated herein by reference.

FIG. 6 is a graph illustrating an imaging sequence which employs a prior art spectral-spatial excitation method. The graph labeled RF includes an RF “fat free” excitation pulse 51. The gradient sequence labeled G_{z} includes a modulated slice selection gradient 53 that is played out during the production of RF excitation pulse 51. The gradient sequences labeled Gₓ and G_{y} are spiral readout gradients 55 and 57. Each of the gradients G_{z}, Gₓ and G_{y} includes a rephasing pulse at the end of the pulse sequence. The frequency of the modulated gradient 53 is calculated so that, when centered on the water resonance, an excitation null occurs at the fat Larmor resonance. In this way only the water Larmor resonance is excited.

FIG. 7 is a graph illustrating a prior art rapid gradient-echo pulse sequence using a spectral-spatial excitation pulse 59. This rf pulse 59 has a gaussian k-space excitation profile that varies along both k_{z} and k_{ω} as disclosed by Meyer et al. The graphs labeled RFI and RFQ represent the real and the imaginary components of this rf excitation pulse 59. The G_{z} gradient sequence 61 is a modulated slice selection gradient and the Gr gradient sequence 63 is a readout gradient. The Ge gradient sequence 65 is a phase encoding gradient. This pulse sequence results in compact spatial and spectral slice profiles (not shown) which are gaussian in shape in the small-tip-angle regime.

Spectral-spatial techniques have the advantage of exciting only the chemical species of interest. Because of this, no presaturation sequence is needed and no sacrifice is necessary on the repetition time (TR). However, spectral-spatial pulses are limited by gradient performance and are especially limited for low field applications where they are prohibitively long in duration. Additionally, careful optimization is required to ensure good spectral selection.

Certain compounds are known to shift the Larmor frequency of spins located in the immediate vicinity of the compound. As disclosed in published US Pat. Appln. No. 2006/0058642 three compounds in the lanthanide family are notable for their chemical shift action. These are dysprosium (Dy), praseodymium (Pr) and europium (Eu) and when administered to a subject under MRI examination, the Larmor frequency of water spin may be shifted away from the Larmor frequency of fat, making the suppression of fat signals much easier to achieve.

### SUMMARY OF THE INVENTION

The present invention is based on the discovery that paramagnetic contrast agents, such as those based on gadolinium, not only shorten the T₁ relaxation of adjacent water spins, but they also produce a chemical shift of the Larmor frequency of those water spins. The invention includes administering such a contrast agent to the subject; acquiring an image using an imaging pulse sequence in which the rf excitation pulse is tuned to the chemically shifted water spins; and interleaving the acquisition with saturation pulse sequences having an rf saturation pulse tuned to the fat and water spins that are not chemically shifted by the contrast agent.

An object of the invention is to acquire contrast enhanced MRA images without the need to subtract a mask image. A gadolinium contrast agent is administered and flows in the subject's blood into the region of interest. The saturation pulse sequences are performed throughout the acquisition at a rate which keeps the signals from tissues surrounding the subject's vasculature suppressed without significantly affecting the signal from blood that contains the contrast agent. In addition, the contrast agent performs its usual function of shortening the T₁ relaxation time of the blood such that many imaging pulse sequences can be performed before the saturated and non-T₁ shortened surrounding tissues recover enough to require another saturation pulse sequence. Thus, the scan is not significantly increased in time.

Another object of the invention is to provide a passive means for tracking devices such as catheters during MRI interventional procedures. The device may be coated with or contain a frequency shifting gadolinium contrast agent and the present invention may be used to produce images in which the signals from water spins surrounding the device are enhanced.

### BRIEF DESCRIPTION OF THE DRAWINGS

FIG. 1 is a block diagram of an MRI system which employs the present invention;

FIGS. 2-7 are graphic representations of prior art methods for selectively suppressing or enhancing tissue signals;

FIG. 8 is a graphic representation of an imaging pulse sequence used with a preferred embodiment of the invention;

FIG. 9 is a graphic representation of a saturation pulse sequence used with the imaging pulse sequence of FIG. 8; and

FIG. 10 is a flow chart of the steps performed by the MRI system of FIG. 1 when practicing the present invention.

### DESCRIPTION OF THE PREFERRED EMBODIMENT

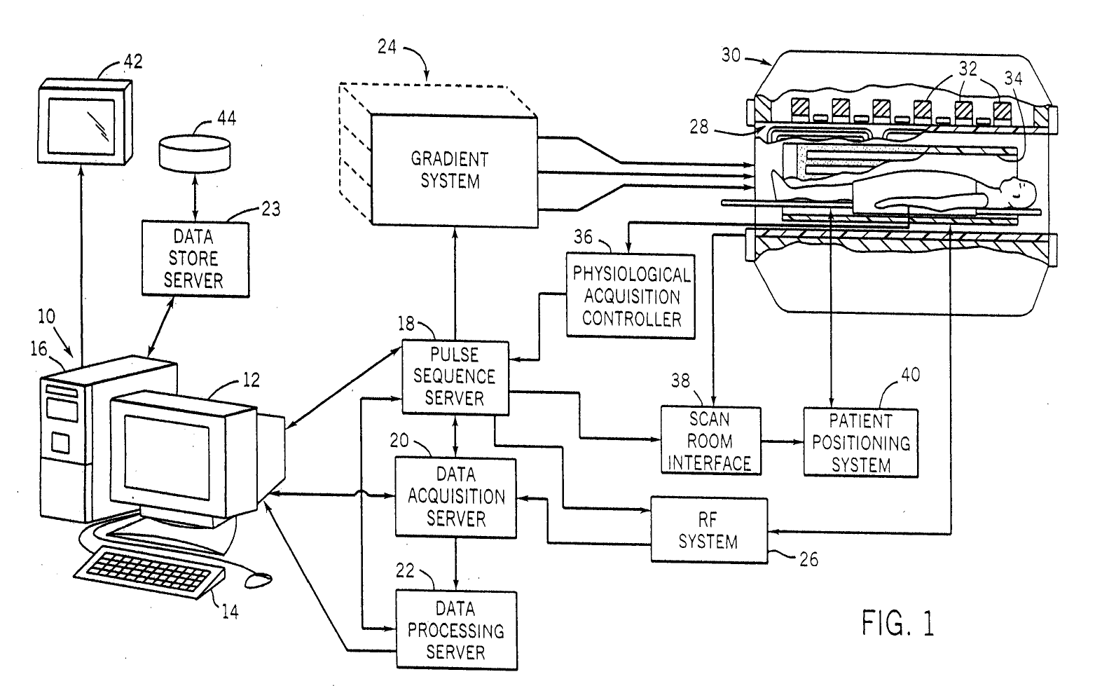

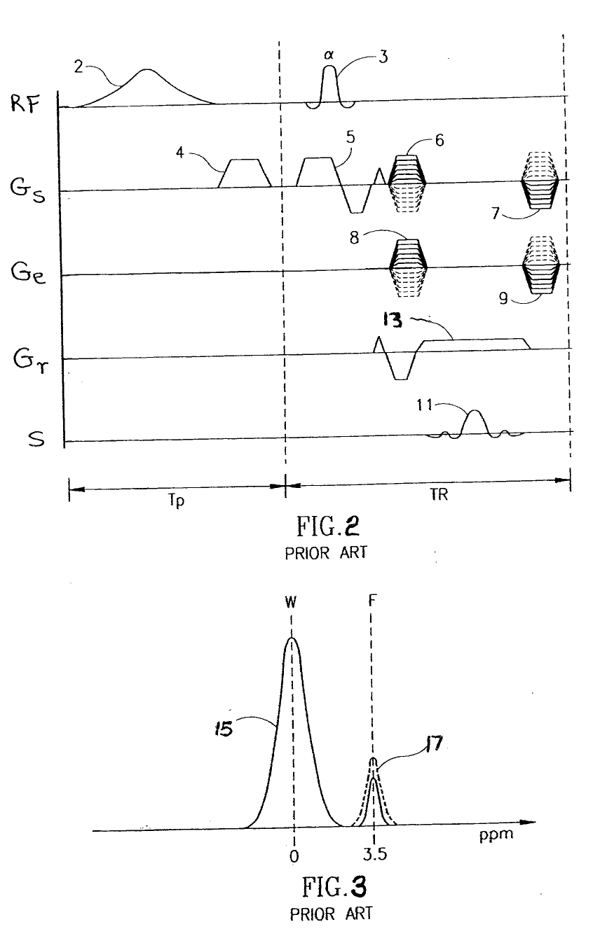

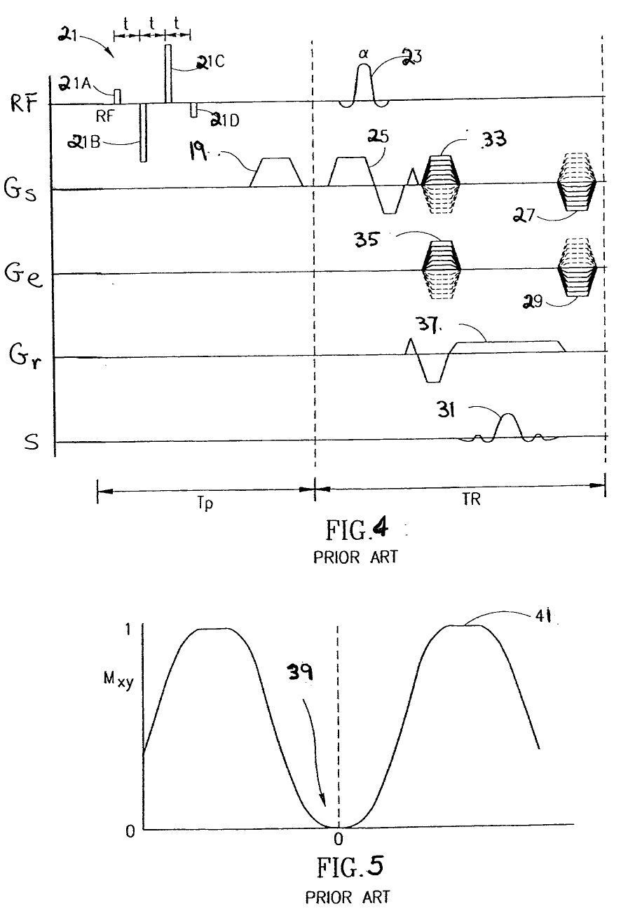

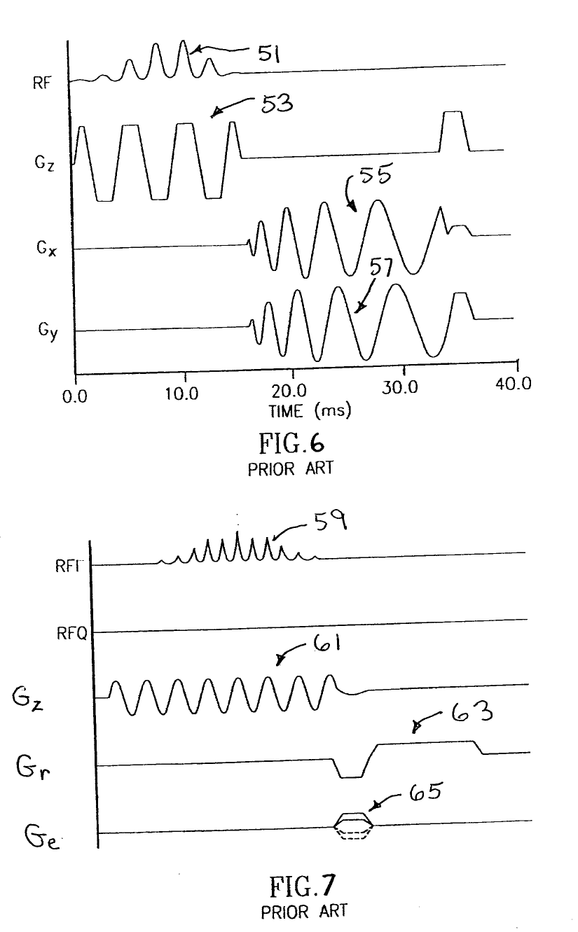

Referring particularly to FIG. 1, the preferred embodiment of the invention is employed in an MRI system. The MRI system includes a workstation 10 having a display 12 and a keyboard 14. The workstation 10 includes a processor 16 which is a commercially available programmable machine running a commercially available operating system. The workstation 10 provides the operator interface which enables scan prescriptions to be entered into the MRI system.

The workstation 10 is coupled to four servers: a pulse sequence server 18; a data acquisition server 20; a data processing server 22, and a data store server 23. In the preferred embodiment the data store server 23 is performed by the workstation processor 16 and associated disc drive interface circuitry. The server 18 is performed by a separate processor and the servers 20 and 22 are combined in a single processor. The workstation 10 and each processor for the servers 18, 20 and 22 are connected to an Ethernet communications network. This network conveys data that is downloaded to the servers 18, 20 and 22 from the workstation 10, and it conveys data that is communicated between the servers.

The pulse sequence server 18 functions in response to instructions downloaded from the workstation 10 to operate a gradient system 24 and an RF system 26. Gradient waveforms necessary to perform the prescribed scan are produced and applied to the gradient system 24 which excites gradient coils in an assembly 28 to produce the magnetic field gradients Gₓ, G_{y} and G_{z} used for position encoding NMR signals. The gradient coil assembly 28 forms part of a magnet assembly 30 which includes a polarizing magnet 32 and a whole-body RF coil 34.

RF excitation waveforms are applied to the RF coil 34 by the RF system 26 to perform the prescribed magnetic resonance pulse sequence. Responsive NMR signals detected by the RF coil 34 are received by the RF system 26, amplified, demodulated, filtered and digitized under direction of commands produced by the pulse sequence server 18. The RF system 26 includes an RF transmitter for producing a wide variety of RF pulses used in MR pulse sequences. The RF transmitter is responsive to the scan prescription and direction from the pulse sequence server 18 to produce RF pulses of the desired frequency, phase and pulse amplitude waveform. The generated RF pulses may be applied to the whole body RF coil 34 or to one or more local coils or coil arrays.

The RF system 26 also includes one or more RF receiver channels. Each RF receiver channel includes an RF amplifier that amplifies the NMR signal received by the coil to which it is connected and a quadrature detector which detects and digitizes the I and Q quadrature components of the received NMR signal. The magnitude of the received NMR signal may thus be determined at any sampled point by the square root of the sum of the squares of the I and Q components:

M=\sqrt{I²+Q²},

and the phase of the received NMR signal may also be determined:

φ=tan⁻¹Q/I.

The pulse sequence server 18 also optionally receives patient data from a physiological acquisition controller 36. The controller 36 receives signals from a number of different sensors connected to the patient, such as ECG signals from electrodes or respiratory signals from a bellows. Such signals are typically used by the pulse sequence server 18 to synchronize, or “gate”, the performance of the scan with the subject's respiration or heart beat.

The pulse sequence server 18 also connects to a scan room interface circuit 38 which receives signals from various sensors associated with the condition of the patient and the magnet system. It is also through the scan room interface circuit 38 that a patient positioning system 40 receives commands to move the patient to desired positions during the scan.

The digitized NMR signal samples produced by the RF system 26 are received by the data acquisition server 20. The data acquisition server 20 operates in response to instructions downloaded from the workstation 10 to receive the real-time NMR data and provide buffer storage such that no data is lost by data overrun. In some scans the data acquisition server 20 does little more than pass the acquired NMR data to the data processor server 22. However, in scans which require information derived from acquired NMR data to control the further performance of the scan, the data acquisition server 20 is programmed to produce such information and convey it to the pulse sequence server 18. For example, during prescans NMR data is acquired and used to calibrate the pulse sequence performed by the pulse sequence server 18. Also, navigator signals may be acquired during a scan and used to adjust RF or gradient system operating parameters or to control the view order in which k-space is sampled. And, the data acquisition server 20 may be employed to process NMR signals used to detect the arrival of contrast agent in an MRA scan. In all these examples the data acquisition server 20 acquires NMR data and processes it in real-time to produce information which is used to control the scan.

The data processing server 22 receives NMR data from the data acquisition server 20 and processes it in accordance with instructions downloaded from the workstation 10. Such processing may include, for example: Fourier transformation of raw k-space NMR data to produce two or three-dimensional images; the application of filters to a reconstructed image; the performance of a backprojection image reconstruction of acquired NMR data; the calculation of functional MR images; the calculation of motion or flow images, etc.

Images reconstructed by the data processing server 22 are conveyed back to the workstation 10 where they are stored. Real-time images are stored in a data base memory cache (not shown) from which they may be output to operator display 12 or a display 42 which is located near the magnet assembly 30 for use by attending physicians. Batch mode images or selected real time images are stored in a host database on disc storage 44. When such images have been reconstructed and transferred to storage, the data processing server 22 notifies the data store server 23 on the workstation 10. The workstation 10 may be used by an operator to archive the images, produce films, or send the images via a network to other facilities.

While the present invention may be used with many different imaging pulse sequences, in the preferred embodiment a three-dimensional, spoiled gradient-echo pulse sequence is used with a sampling bandwidth of 83-125 kHz during an MRA examination of a subject. Referring to FIG. 8, this preferred imaging pulse sequence employs a spectral-spatial excitation 220 in the presence of gradient 222 to produce transverse magnetization in the 3D volume of interest. In the alternative, a spectrally-selective, spatially non-selective rf excitation pulse may be used. In either case, the rf excitation pulse 220 is followed by a phase encoding gradient pulse 224 directed along the z axis and a phase encoding gradient pulse 226 directed along the y axis. A readout gradient pulse 228 directed along the x axis follows and a partial echo (60%) NMR signal 230 is acquired and digitized as described above. After the acquisition, rewinder gradient pulses 232 and 234 rephase the magnetization before the pulse sequence is repeated to acquire another view at a different phase encoding. As is well known in the art, the pulse sequence is repeated and the phase encoding pulses 224 and 226 are stepped through a series of values to sample the 3D k-space.

Sampling along the kₓ axis is performed by sampling the echo signal 230 in the presence of the readout gradient pulse 228 during each pulse sequence. It will be understood by those skilled in the art that only a partial sampling along the kₓ axis is performed and the missing data is computed using a homodyne reconstruction or by zero filling. This enables the echo time (TE) of the pulse sequence to be shortened to 1-2 ms and the pulse repetition rate (TR) to be shortened to 3 to 6 msecs.

The rf excitation pulse 220 in this imaging pulse sequence is tuned to excite water spins that have been chemically shifted by an administered paramagnetic contrast agent. The amount of this chemical shift depends on the type and concentration of the contrast agent, but in the preferred embodiment gadolinium-DTPA is used and typical frequency shifts are given in Table 1.

TABLE 1

| B₀ | Chemical Shift | |
|---|---|---|
| 1.5 T | 225 | Hz |
| 3.0 T | 450 | Hz |
| 7.0 T | 1050 | Hz |

The excitation pulse 220 may also be spectrally selective to the off-resonance water spins. A number of different approaches can be used to provide spectral selectively, but because the TE and TR times of the imaging pulse sequence must be kept very short, the effectiveness of spectral selectivity is limited.

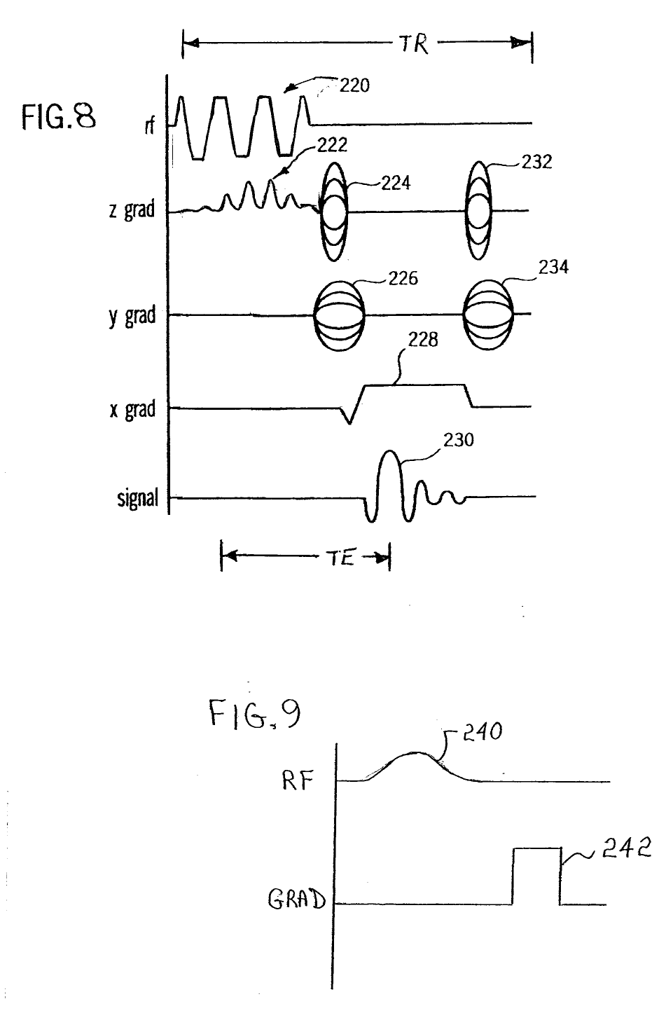

The suppression of signals from background tissues is achieved primarily by interleaving a saturation pulse sequence with the image pulse sequence repetitions. Referring to FIG. 9, the saturation pulse sequence is comprised of a spectrally selective rf saturation pulse 240, followed by a spoiler gradient pulse 242. The saturation pulse 240 in this preferred embodiment is a frequency selective gaussian saturation pulse with the flip angle of 110° that is tuned to a frequency midway between the Larmor frequency of fat and the Larmor frequency of on-resonance water. Since the saturation pulse sequence is performed far less often than the imaging pulse sequence, the duration of the rf saturation pulse 240 is not as critical and it may be extended to insure adequate selection of on-resonance, background tissues without substantially affecting the off-resonance water spins. The spoiler gradient 242 dephases the transverse magnetization produced by the saturation pulse 240.

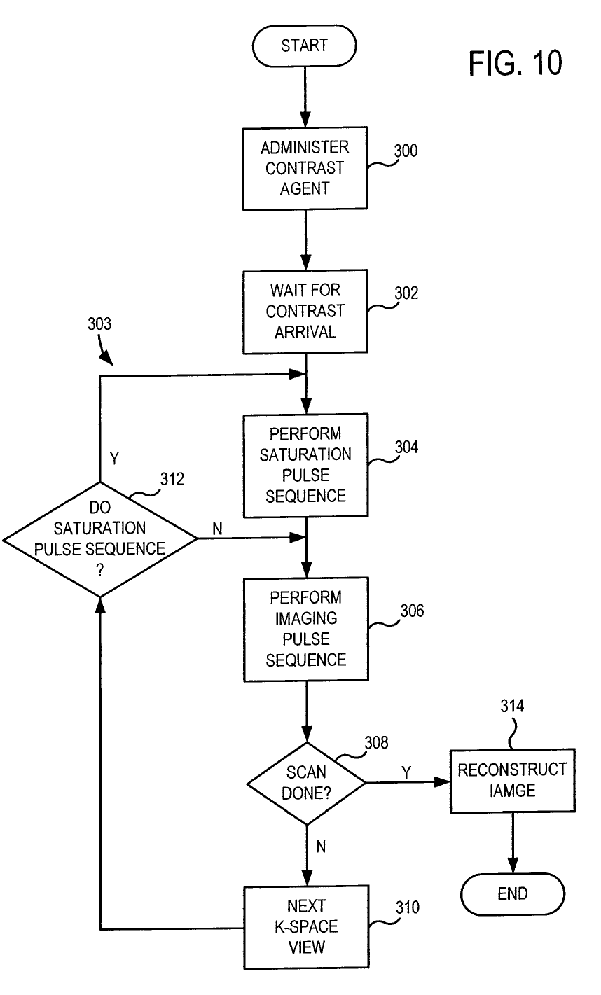

Referring particularly to FIG. 10, a scan is conducted to acquire one or more images using the imaging pulse sequence of FIG. 8 and the saturation pulse sequence of FIG. 9. As indicated at process block 300, a contrast agent is first administered and allowed to circulate into the vasculature of interest. A contrast agent that shortens The T1 relaxation time, such as a gadolinium chelate or ultra-small particles of iron oxide (USPIO), is administered. For gadolinium-enhanced MRA, at least 0.2 mmol/kg (body weight) of gadolinium chelate is administered intravenously at a rate of at lease 3 cc/sec. The system pauses until the contrast agent enters the vasculature that is to be imaged as indicated at process block 302 and a loop indicated generally at 303 is then entered in which the image data is acquired.

As indicated at process block 304, the above-described saturation pulse sequence is performed to saturate the on-resonance spins in the region of interest. Then the above-described imaging pulse sequence is performed as indicated at process block 306 to acquire a single view of k-space data. A check is then made at decision block 308 to determine if all the k-space data for the prescribed image (or images) have been acquired. If not, the gradients for the next view are determined as indicated at process block 310 and the system loops back to acquire the next view.

Before acquiring the next view, however, a check is made at decision block 312 to determine if the saturation pulse sequence should be performed first. Typically, from 4 to 128 imaging pulse sequences can be performed before the longitudinal magnetization of surrounding on-resonance background tissues recovers from the previous saturation pulse sequence. The exact number of views acquired between saturation pulse sequences forms part of the scan prescription and from 48 to 128 views may be acquired before another saturation pulse sequence is produced. Either the imaging pulse sequence is performed, or if the prescribed number of image views have been acquired, the saturation pulse sequence is performed again at process block 304.

When all the image data has been acquired as determined at decision block 308, the acquired k-space data is used to reconstruct one or more images as indicated at process block 314. A conventional image reconstruction method is used such as a 3DFT.

It should be apparent that many different imaging pulse sequences can be employed without departing from the spirit of the invention. An important factor, however, is that the rf excitation pulses used in the imaging pulse sequence be tuned to the off-resonance frequency of the contrast enhanced spins. Also, various types of known spectrally selective rf pulses may be used in both the imaging pulse sequence and the saturation pulse sequence.

The most common clinical application of the present invention is MRA where the contrast agent is injected into the vasculature of the subject being examined. However, there are other clinical applications where the present invention may be employed.

In MRI intervention procedures devices such as catheters are used to perform a procedure on the subject. MR images are acquired as the procedure is performed in order to help guide the device into proper position. To accomplish this the device must be clearly visible in the MR image. By coating the device, or portions of the device, with contrast agent or otherwise carrying contrast agent on the device, the surrounding water spins are chemically shifted and will be clearly seen on an image acquired according to the present invention. The device is thus brightly outlined in the MR image so that its location can be clearly seen. In this clinical application, therefore, the contrast agent is administered by insertion of the device into the subject under examination.

1. A method for suppressing signals from background tissues during the acquisition of image data with a magnetic resonance imaging (MRI) system, the steps comprising:
administering a contrast agent to the subject that shortens the T₁ relaxation time of adjacent water spins and that shifts the Larmor frequency of said adjacent water spins;
acquiring image data from the subject using an imaging pulse sequence having an rf excitation pulse tuned to the shifted Larmor frequency of said adjacent water spins; and
interleaving saturation pulse sequences with the imaging pulse sequences, the saturation pulse sequence including an rf saturation pulse selectively tuned to saturate one of fat spins and water spins whose Larmor frequency has not been shifted by the contrast agent.

2. The method as recited in claim 1 in which the saturation pulse sequence also includes applying a spoiler gradient pulse to dephase transverse magnetization produced by the rf saturation pulse.

3. The method as recited in claim 1 in which the rf excitation pulse in the imaging pulse sequence is a spectrally selective rf excitation pulse.

4. The method as recited in claim 1 in which a plurality of imaging pulse sequences are performed following each saturation pulse sequence.

5. The method as recited in claim 1 in which the rf excitation pulse has a first frequency selective band, the rf saturation pulse has a second frequency selective band, and the first and second bands do not substantially overlap.

6. The method as recited in claim 1 in which the contrast agent is administered by injecting it into the vasculature of the subject.

7. The method as recited in claim 6 in which the contrast agent contains one of Gadolinium and iron.

8. The method as recited in claim 1 in which the contrast agent is supported by a device and the device is inserted into the subject.

9. The method as recited in claim 8 in which the contrast agent contains one of Gadolinium and iron.

10. The method as recited in claim 8 in which the device is a catheter.
